(19) United States
(12) Patent Application Publication
(10) Pub. No.: US 2011/0098470 A1
(43) Pub. Date: Apr. 28, 2011

(54) METHOD FOR PRODUCING THE TRISODIUM SALT OF 2,4,6-TRIMERCAPTO-S-TRIAZINE

(75) Inventors: Joerg Grimmer, Seligenstadt (DE); Ruediger Peldszus, Hasselroth (DE); Peter Verelst, Burcht (BE); Roger Vandervee, Maaseik (BE)

(73) Assignee: EVONIK DEGUSSA GMBH, ESSEN (DE)

(21) Appl. No.: 12/993,705

(22) PCT Filed: Apr. 22, 2009
(86) PCT No.: PCT/EP2009/054807

§ 371 Date: Nov. 19, 2010

(30) Foreign Application Priority Data
May 29, 2008 (DE) 102008002073.7

(51) Int. Cl. C07D 251/38

(52) U.S. Cl. 544/219

(57) ABSTRACT

The invention relates to a method for producing the trisodium salt of 2,4,6-trimercapto-s-triazine, cyanuric chloride being reacted in an aqueous medium at a temperature of 20 to 70° C. using NaSH, Na₂S or a mixture of the two compounds at a pH value of 7 to 11, and subsequently the trisodium salt of 2,4,6-trimercapto-s-triazine being precipitated and cleaved at a pH value of 0.12 and a temperature of 0 to 20° C., characterized in that after the cleavage step of the trisodium salt of 2,4,6-trimercapto-s-triazine the mother liquor is adjusted to a pH value of 6 to 8, wherein the monosodium salt of 2,4,6-trimercapto-s-triazine is precipitated.

The present invention describes a process for preparing the trisodium salt of 2,4,6-trimercapto-s-triazine (TMT-Na₃).

The preparation of the trisodium salt of 2,4,6-tri-mercapto-s-triazine (TMT-Na₃>9 H₂O) is described in DE 37 29 029. Cyanuric chloride is firstly reacted in an aqueous medium with NaSH, Na₂S or a NaSH/Na₂S mixture at temperatures of from 20 to 70° C., with a pH of >7 being set. After the reaction, the reaction mixture obtained is cooled to from 0 to 20° C. and the trisodium salt (TMT-Na₃) which has crystallized out is filtered off. This filtrate is set to a pH of from 2 to 4 by means of hydrochloric acid, the 2,4,6-trimercapto-s-triazine (TMT-H₃) which now precipitates is separated off and introduced without drying into a sodium hydroxide solution. This gives an aqueous 30-40% strength by weight solution of the trisodium salt (TMT-Na₃) which is subsequently cooled to from 0 to 20° C., so that the trisodium salt (TMT-Na₃) now crystallizes out and can be separated off. The filtrate now obtained is fed to the reactor in which the reaction of the cyanuric chloride takes place.

EP 0 413 980 A1 describes a similar preparative process but in this case for the preparation of the disodium salt of 2,4,6-trimercapto-s-triazine (TMT-Na₂×6 H₂O), in which, for the work-up, the filtrate and the washing water after the crystallized trisodium salt (TMT-Na₃) has been separated off are also acidified with hydrochloric acid and the precipitated 2,4,6-tri-mercapto-s-triazine (TMT-Na₃) is separated off. This separated-off 2,4,6-trimercapto-s-triazine (TMT-H₃) and also the separated-off trisodium salt (TMT-Na₃) are then stirred into water. This aqueous solution is evaporated and cooled to 10° C., resulting in crystallization of a solid which is subsequently separated off. Careful drying gives the disodium salt of 2,4,6-trimercapto-s-triazine (TMT-Na₂).

A process for preparing the trisodium salt of 2,4,6-trimercapto-s-triazine (TMT-Na₃) is likewise described by Liu et al., in Wuhan Huagong Xueyuan Xuebao (2005), 27(4), 8-10. In contrast to the above processes, the reaction mixture is not cooled but instead the undissolved solids are filtered off, hydrochloric acid is added and the precipitate which forms (TMT-H₃) is likewise separated off. The 2,4,6-trimercapto-s-triazine (TMT-H₃) is subsequently recrystallized in methanol. This 2,4,6-trimercapto-s-triazine (TMT-H₃) is then introduced into an aqueous sodium hydroxide solution and the insoluble constituents are filtered off. The pH is set to 12-12.5; a 15% strength solution of the trisodium salt of 2,4,6-trimercapto-s-triazine (TMT-Na₃) is obtained.

The Korean patent document KR 10-0695193 describes a process for preparing alkali metal salts of 2,4,6-tri-mercapto-s-triazine; here, the reaction solution obtained is set to a pH of 6 or less by addition of hydrochloric acid, resulting in 2,4,6-trimercapto-s-triazine (TMT-H₃) settling out. This is separated off, dispersed in water and admixed with sodium hydroxide, so that the trisodium salt of 2,4,6-trimercapto-s-triazine (TMT-Na₃) is formed. The solution is evaporated and cooled to from 0 to 20° C., the trisodium salt (TMT-Na₃) is separated off and the filtrate is recirculated to the process.

U.S. Pat. No. 5,563,267 likewise describes a process for preparing trialkali metal salts of trimercapto-s-triazine in an acetone/water mixture. The reaction mixture is cooled to room temperature and admixed with an acid. The resulting precipitate is separated off and introduced into a basic solution. The precipitate which now forms (trialkali metal salt of trimercapto-s-triazine) is separated off.

A process for preparing 2,4,6-trimercapto-s-triazine (TMT-H₃) is described in DE 10 2005 036 693 A1; here, the 2,4,6-trimercapto-s-triazine (TMT-H₃) is precipitated at a pH of from 1.5 to 2.5.

It was an object of the present invention to provide a process for preparing the trisodium salt of 2,4,6-tri-mercapto-s-triazine, which makes it possible to reduce the consumption of acids and bases while maintaining a yield comparable to that in the prior art.

We have surprisingly found a process for preparing the trisodium salt of 2,4,6-trimercapto-s-triazine (TMT-Na₃), which is characterized in that the mother liquor after the trisodium salt of 2,4,6-trimercapto-s-triazine (TMT-Na₃) has been separated off is set to a pH of from 5.5 to 8. In contrast thereto, in processes according to the prior art, the mother liquor is set to a pH of from 2 to 4, as described, for example, in DE 37 29 029 A1. The lowering of the pH from 12 to a pH of from 5.5 to 8 results in a significantly smaller amount of acid being necessary than in a process which requires a pH of less than 4 immediately after the trisodium salt of 2,4,6-trimercapto-s-triazine (TMT-Na3) has been separated off. At a pH of from 5.5 to 8, the 2,4,6-trimercapto-s-triazine compounds still present in the mother liquor can likewise be precipitated, but as monosodium salt (TMT-Na₁) instead of in the form of 2,4,6-trimercapto-s-triazine (TMT-H₃). In the process of the invention, the monosodium salt (TMT-Na₁) which has been precipitated and separated off from the mother liquor is redissolved by increasing the pH. This pH is preferably the same pH as is set in the reaction of the cyanuric chloride. In this way, this solution can be recirculated to the reactor after the reaction but before precipitation of the trisodium salt (TMT-Na₃). In processes according to the prior art, the 2,4,6-tri-mercapto-s-triazine (TMT-H₃) is introduced into sodium hydroxide solution in order to be able to precipitate the trisodium salt (TMT-Na₃) again. Since the trisodium salt (TMT-Na₃) generally precipitates at a pH of 12, it is necessary to increase the pH from 2-4 to 12. Considerable amounts of acids and bases are therefore necessary in the processes according to the prior art. In contrast, the pH changes in the process of the invention are smaller and less acid or base is therefore required. It was also surprising that not only is there a saving of acid and base but the total yield of trisodium salt (TMT-Na₃) is comparable to the total yield of processes according to the prior art.

A further advantage of the process of the invention is the possibility of isolating the hydrogen sulfide formed in the reaction of the cyanuric chloride, reacting it with sodium hydroxide and recirculating it to the reaction. In this way, the economics and environmental friendliness of the process of the invention can be improved further.

The invention accordingly provides a process for preparing the trisodium salt of 2,4,6-trimercapto-s-triazine (TMT-Na₃), where cyanuric chloride is reacted in an aqueous medium with NaSH, Na₂S or a mixture of these two compounds at a temperature of from 20 to 70° C. and a pH of from 7 to 11 and the trisodium salt of 2,4,6-trimercapto-s-triazine (TMT-Na₃) is subsequently precipitated and separated off at a pH of 12 and a temperature of from 0 to 20° C., which is characterized in that the mother liquor after the trisodium salt of 2,4,6-trimercapto-s-triazine (TMT-Na₃) has been separated off is set to a pH of from 6 to 8, resulting in the monosodium salt of 2,4,6-trimercapto-s-triazine (TMT-Na₁) precipitating.

In the reaction of the cyanuric chloride in the process of the invention, NaSH and/or Na₂S are preferably used as sulfur-containing component. It is also possible to use a mixture of NaSH and Na₂S, preferably in a molar ratio of Na₂S to NaSH of from 1:1 to 1:4, in particular from 1:2 to 1:4. It is also possible to use an aqueous solution of NaSH, Na₂S and sodium hydroxide, as is formed in the scrubbing of hydrogen sulfide gas by means of sodium hydroxide, as further sulfur-containing component. Preference is given to using both NaSH and/or Na₂S and also an aqueous solution of NaSH, Na₂S and sodium hydroxide. The molar ratio of the total amount of sulfur to cyanuric chloride in the reaction is preferably at least 3:1, but more preferably from 3.5:1 to 9:1 and particularly preferably from 4:1 to 8:1. The reaction of the cyanuric chloride in the process of the invention is preferably carried out at a pH of from 9 to 11, more preferably from 10 to 11. The temperature here is preferably from 40 to 60° C., more preferably from 45 to 55° C. and particularly preferably 50° C. After the reaction or addition of the starting materials, an after-reaction can be carried out in the process of the invention at the same temperature and at the same pH as in the main reaction.

For the trisodium salt of 2,4,6-trimercapto-s-triazine (TMT-Na₃) to be precipitated virtually completely, the pH of the reaction solution is preferably set to 12, preferably to a pH of from 12 to 13.5, after the reaction or after the after-reaction of the process of the invention. Furthermore, it is advisable to cool the reaction solution to a temperature of from 5 to 30° C., preferably from 10 to 20° C. The trisodium salt of 2,4,6-trimercapto-s-triazine (TMT-Na₃) which has been precipitated in this way can then be separated off by means of a separation process; the trisodium salt is preferably separated off in the process of the invention by means of a mechanical separation process, for example filtration, centrifugation or decantation.

The filtrate or the aqueous phase present after the trisodium salt (TMT-Na₃) has been separated off is referred to as mother liquor for the purposes of the present invention. This mother liquor is preferably set to a pH of from 6 to 8, more preferably to a pH of from 6.5 to 7.5 and particularly preferably to a pH of 7, in the process of the invention. The pH can be adjusted by means of an inorganic acid, preferably by means of hydrochloric acid and particularly preferably by means of a 10-25% strength, very particularly preferably 16-22% strength, hydrochloric acid. At this pH, the monosodium salt of 2,4,6-trimercapto-s-triazine (TMT-Na₁), which like 2,4,6-trimercapto-s-triazine (TMT-H₃) has a low solubility in water, can be precipitated. This is preferably carried out at a temperature of from 10 to 45° C., more preferably at a temperature of from 30 to 45° C.

In the process of the invention, the precipitated monosodium salt of 2,4,6-trimercapto-s-triazine (TMT-Na₁) can subsequently be separated off from the mother liquor by means of a separation process, preferably by means of a mechanical separation process, for example filtration, centrifugation or decantation.

The monosodium salt of 2,4,6-trimercapto-s-triazine (TMT-Na₁) which has been separated off can, in the process of the invention, then be brought into solution in an aqueous medium at a pH of from 9 to 11, preferably at a pH of from 10 to 11. Sodium hydroxide, preferably a 5-60% strength, particularly preferably 30-55% strength, sodium hydroxide solution is preferably used for this purpose. As temperature, it is possible to set a temperature of from 20 to 50° C. The monosodium salt of 2,4,6-trimercapto-s-triazine (TMT-Na₁) is preferably brought into solution by means of sodium hydroxide at a pH of from 10 to 11 and a temperature of from 20 to 50° C., forming the disodium salt of 2,4,6-trimercapto-s-triazine (TMT-Na₂).

This aqueous solution of the disodium salt of 2,4,6-trimercapto-s-triazine (TMT-Na₂) can then be recirculated to the reactor, preferably after the reaction and after-reaction of the cyanuric chloride are concluded, but preferably before precipitation of the trisodium salt (TMT-Na₃).

The pH of the filtrate from the isolation of the monosodium salt of 2,4,6-trimercapto-s-triazine (TMT-Na₁) can then be reduced further, preferably to a pH of from 0 to 4 and more preferably to a pH of from 1.5 to 2.5. At this pH, the 2,4,6-trimercapto-s-triazine (TMT-H₃) can then be precipitated and separated off by means of a separation process. This precipitated 2,4,6-trimercapto-s-triazine (TMT-H₃) can be recirculated to the work-up process for the mother liquor; it is preferably added to the mother liquor before precipitation of the monosodium salt of 2,4,6-trimercapto-s-triazine (TMT-Na₁).

As a result of the reaction of cyanuric chloride with NaSH, Na₂S or a mixture of these two compounds, hydrogen sulfide is liberated or is present in the aqueous solution. For environmental protection reasons and also to increase the purity of the target product, the hydrogen sulfide formed is preferably collected and passed through one or more gas scrubbers containing sodium hydroxide. This sodium hydroxide which has been reacted with hydrogen sulfide can then be recirculated as starting material to the reactor. It is advantageous to apply a subatmospheric pressure of <1000 mbar, preferably a pressure of from 150 to 250 mbar, to the mother liquor before precipitation of the monosodium salt of 2,4,6-trimercapto-s-triazine (TMT-Na₁). The pressure and the temperature should be selected so that very little water goes into the gas phase, and the mother liquor is therefore preferably degassed at a temperature of from 10 to 55° C., preferably from 30 to 45° C. The gases given off are then preferably passed through one or more gas scrubbers containing sodium hydroxide. The aqueous solution of NaSH, Na₂S and sodium hydroxide formed by reaction of hydrogen sulfide and sodium hydroxide can then be recirculated to the reactor which serves for the reaction of cyanuric chloride with NaSH and/or Na₂S.

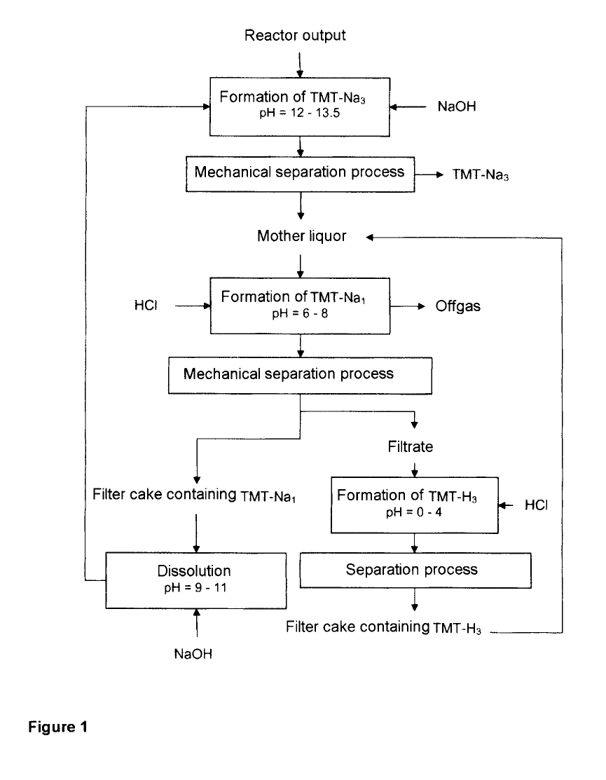

FIG. 1 shows a process flow diagram depicting the preferred process steps and streams of the process of the invention.

The following examples illustrate the process of the invention without restricting the invention to this embodiment.

### EXAMPLE 1

2.1 1 of a 40% strength by weight NaSH solution are placed in a reactor and 1.5 1 of a 50% strength by weight sodium hydroxide solution and an aqueous cyanuric chloride suspension (2.7 kg of cyanuric chloride in 4.5 1 of water) are added at a pH of 10.5 and a temperature of 50° C. After the addition, the reaction mixture is allowed to react for a further 30 minutes. The pH is set to 12.5-13 by means of a 50% strength by weight sodium hydroxide solution. The reaction mixture is allowed to cool to 20° C. The TMT-Na₃ which forms is then separated off from the mother liquor by means of a centrifuge.

### EXAMPLE 2

941.2 g of the mother liquor produced in example 1 are set to pH=2 by means of 227.6 g of a 16.22% strength by weight hydrochloric acid. The mother liquor is degassed at a pressure of 200 mbar and a temperature of 40° C.; a mixture of hydrogen sulfide and water is given off. At the same time, TMT-H₃ is precipitated; this is allowed to settle and subsequently separated off from the filtrate. This filter cake (48.8 g) is admixed with 63.0 g of a 19.75% strength by weight sodium hydroxide solution and 71.8 g of water, so that a pH of 12.5 is obtained. The TMT-Na₃ then precipitates at a temperature of 40-45° C., and is once again separated from the filtrate by filtration at a temperature of 20° C.

The consumption of HCl is 1.025 mol and that of NaOH is 0.311 mol.

### EXAMPLE 3

941.7 g of the mother liquor produced in example 1 are set to pH=7 by means of 164.4 g of a 16.22% strength by weight hydrochloric acid. The mother liquor is degassed at a pressure of 200 mbar and a temperature of 40° C.; a mixture of hydrogen sulfide and water is given off. At the same time, TMT-Na₁ precipitates; this is allowed to settle and subsequently filtered off from the filtrate. The filtrate is then brought to pH=2 by means of 47.7 g of a 16.22% strength by weight hydrochloric acid. At a temperature of 40° C., TMT-H₃ then precipitates and is separated off by means of settling and filtration. The filter cake containing TMT-Na₁ (40.5 g) and the filter cake containing TMT-H₃ (10.7 g) are combined and admixed with 46.7 g of a 19.75% strength by weight sodium hydroxide solution and 48.8 g of water, so that a pH of 12.5 is obtained. The TMT-Na₃ then precipitates at a temperature of 40-45° C. and is once again separated from the filtrate by means of filtration at a temperature of 20° C.

The consumption of HCl is (0.741+0.215) mol=0.956 mol and that of NaOH is 0.230 mol.

The two examples 2 and 3 show that the consumption of hydrochloric acid and sodium hydroxide and thus the sodium chloride burden arising in the plant is lower in example 3.

### EXAMPLE 4

The mother liquor produced in example 1 is set to pH=7 by means of a 12% strength by weight hydrochloric acid. The mother liquor is degassed at a pressure of 200 mbar and a temperature of 45° C.; a mixture of hydrogen sulfide and water is given off. At the same time, TMT-Na₁ precipitates, and this is allowed to settle and the filtrate is subsequently decanted off.

The filter cake containing TMT-Na₁ is admixed with 50% strength by weight sodium hydroxide solution and the solution obtained is set to a pH of 10.5. The solution obtained is fed to the reactor outlet before precipitation of the TMT-Na₃.

The filtrate from the isolation of the TMT-Na₁ is brought to pH=2 by means of a 12% strength by weight hydrochloric acid. TMT-H₃ precipitates and is separated off by means of settling and decantation and added to the mother liquor from example 1.

In this way, the total yield can be increased to 92.9% based on the cyanuric chloride used.

1. A process for preparing the trisodium salt of 2, 4, 6-trimercapto-s-triazine (TMT-Na₃), the process comprising:
(A) reacting cyanuric chloride in an aqueous medium with at least one selected from the group consisting of NaSH and Na₂S, at a temperature of from 20 to 70° C. and a pH of from 7 to 11; and
(B) subsequently precipitating and separating off a trisodium salt of 2, 4, 6-trimercapto-s-triazine (TMT-Na₃) from a mother liquor at a pH of 12 and a temperature of from 0 to 20° C.; and
(C) setting the mother liquor, after the trisodium salt of 2, 4, 6-trimercapto-s-triazine (TMT-Na₃) has been separated off, to a pH of from 6 to 8, and thereby precipitating a monosodium salt of 2, 4, 6-trimercapto-s-triazine (TMT-Na₁).

2. The process as claimed in claim 1, wherein the mother liquor is set to a temperature of from 10 to 45° C.

3. The process as claimed in claim 1, wherein the monosodium salt of 2, 4, 6-trimercapto-s-triazine (TMT-Na₁) precipitated in the setting (C) is separated off from the mother liquor by a mechanical separation process.

4. The process as claimed in claim 3, wherein the pH of a filtrate from isolating the monosodium salt of 2, 4, 6-trimercapto-s-triazine (TMT-Na₁) after precipitating it in the setting (C) is set to a pH of from 1.5 to 2.5.

5. The process as claimed in claim 4, wherein 2, 4, 6-trimercapto-s-triazine (TMT-H₃), which precipitates upon lowering the pH of the filtrate, is separated off by a separation process, and the 2, 4, 6-trimercapto-s-triazine (TMT-H₃) obtained is added to the mother liquor before precipitation of the monosodium salt of 2, 4, 6-trimercapto-s-triazine (TMT-Na₁).

6. The process as claimed in claim 3, wherein the monosodium salt of 2, 4, 6-trimercapto-s-triazine (TMT-Na₁) separated off from the mother liquor is brought into solution by contacting it with a sodium hydroxide solution at a pH of from 10 to 11 and a temperature of from 20 to 50° C., forming an aqueous solution of a disodium salt of 2, 4, 6-trimercapto-s-triazine (TMT-Na₂).

7. The process as claimed in claim 6, the aqueous solution of the disodium salt of 2, 4, 6-trimercapto-s-triazine (TMT-Na₂) is recirculated a reactor, wherein the reacting (A) is carried out.

8. The process as claimed in claim 1, wherein a subatmospheric pressure of <1000 mbar is applied to the mother liquor in the setting (C) before precipitation of the monosodium salt of 2, 4, 6-trimercapto-s-triazine (TMT-Na₁).

9. The process as claimed in claim 8, wherein the mother liquor is degassed under the subatmospheric pressure at a temperature of from 10° C. to 55° C.

10. The process as claimed in claim 8, wherein gases given off under the subatmospheric pressure are passed through at least one gas scrubber comprising sodium hydroxide.

11. The process as claimed in claim 10, wherein an aqueous solution comprising NaSH, Na₂S, and sodium hydroxide from the at least one gas scrubber is recirculated to a reactor, wherein the reacting (A) is carried out.

12. The process as claimed in claim 2, wherein the monosodium salt of 2, 4, 6-trimercapto-s-triazine (TMT-Na₁) precipitated in the setting (C) is separated off from the mother liquor by a mechanical separation process.

13. The process as claimed in claim 12, wherein the pH of a filtrate from isolating the monosodium salt of 2, 4, 6-trimercapto-s-triazine (TMT-Na₁) after precipitating it in the setting (C) is set to a pH of from 1.5 to 2.5.

14. The process as claimed in claim 13, wherein 2, 4, 6-trimercapto-s-triazine (TMT-H₃), which precipitates upon lowering the pH of the filtrate, is separated off by a separation process, and the 2, 4, 6-trimercapto-s-triazine (TMT-Na₃) obtained is added to the mother liquor before precipitation of the monosodium salt of 2,4,6-trimercapto-s-triazine (TMT-Na₁).

15. The process as claimed in claim 12, wherein the monosodium salt of 2, 4, 6-trimercapto-s-triazine (TMT-Na₁) separated off from the mother liquor is brought into solution by contacting it with a sodium hydroxide solution at a pH of from 10 to 11 and a temperature of from 20 to 50° C., forming an aqueous solution of a disodium salt of 2, 4, 6-trimercapto-s-triazine (TMT-Na₂).

16. The process as claimed in claim 15, the aqueous solution of the disodium salt of 2, 4, 6-trimercapto-s-triazine (TMT-Na₂) is recirculated to a reactor, wherein the reacting (A) is carried out.

17. The process as claimed in claim 9, wherein gases given off in the degassing are passed through at least one gas scrubber containing sodium hydroxide.

18. The process as claimed in claim 10, wherein an aqueous solution of NaSH, Na₂S, and sodium hydroxide from the at least one gas scrubber is recirculated to a reactor, wherein the reacting (A) is carried out.
